(19) United States
(12) Patent Application Publication
(10) Pub. No.: US 2013/0072466 A1
(43) Pub. Date: Mar. 21, 2013

(54) COMPOSITION CONTAINING PLACENTA EXTRACTS

(71) Applicants: In Ho Choi, Gyeongsan-si (KR); Dong Mok Lee, Daegu (KR); Eun Ju Lee, Daegu (KR); Ki-Ho Lee, Seoul (KR); Yong-Pil Cheon, Seoul (KR); Tae-Hoon Chun, Seoul (KR)

(72) Inventors: In Ho Choi, Gyeongsan-si (KR); Dong Mok Lee, Daegu (KR); Eun Ju Lee, Daegu (KR); Ki-Ho Lee, Seoul (KR); Yong-Pil Cheon, Seoul (KR); Tae-Hoon Chun, Seoul (KR)

(73) Assignee: Industry-Academic Cooperation Foundation, Yeungnam University, Gyeongsan-si, Gyeongsangbuk-do (KR)

(21) Appl. No.: 13/580,242

(22) PCT Filed: Feb. 21, 2011
(86) PCT No.: PCT/KR2011/001110

§ 371 Date: Dec. 6, 2012

(30) Foreign Application Priority Data
Feb. 22, 2010 (KR) 10-2010-0015907
Dec. 28, 2010 (KR) 10-2010-0136550
Dec. 28, 2010 (KR) 10-2010-0136551

(51) Int. Cl. A61K 31/5685; A61K 31/565; A61K 31/568

(52) U.S. Cl. CPC A61K 31/5685; A61K 31/568; A61K 31/565
USPC 514/181; 514/178; 514/177; 514/182

(57) ABSTRACT

Provided is a composition containing placenta extracts as active ingredients. Placenta extracts are natural substances extracted from placentas of livestock and show effects that can replace steroids and reduce adverse effects of steroids, so that placenta extracts have a wide range of applications including contraceptives, anti-osteoporosis drugs, anti-anemic drugs, therapeutic agents for wasting diseases of muscular atrophy, agents for treating sexual dysfunction, therapeutic agents for wounds, and adipocyte differentiation stimulating agents for improving meat quality of livestock, etc.

### TECHNICAL FIELD

The present invention relates to a composition containing as an active steroid ingredient a placenta extract from currently discarded placenta of a mammal such as a swine that is a natural substance and is known to have muscle strengthening effects, growth promotion effects, and disease therapeutic effects, and a method of preparing the placenta extract.

### BACKGROUND ART

Anabolic steroid is defined as a testosterone derivative synthesized by changing a chemical structure of testosterone. This is known as a hormone that has a stronger effect than naturally existing testosterone. However, when excessively used, men's reproductive functions decrease and cardiovascular disorder may occur. Due to such side effects, the Olympic committee in 1964 prohibited sports players from taking anabolicsteroid.

In addition, although testosterone is administered to sexually dysfunctional men, testosterone may cause prostate cancer due to the conversion into dihydrotestosterone by 5α-reducing enzyme.

Meanwhile, placenta is a nutrition supplier that absorbs blood, rich in nutrition and oxygen, from a uterine wall of a mother body and supplies it to a fetus through an umbilical cord. Placenta is also rich in other nutrients including amino acids that are required for a human body and an active peptide that aid pharmaceutical activities, vitamins, nucleic acids, and enzymes. In addition, placenta further includes various growth factors, such as a hepatocyte growth factor, a neuron growth factor, an epithelial growth factor, a fibroblast growth factor, an insulin-like growth factor, or a growth factor for enhancing immunity.

Up to now, protein, lipid, nucleic acids, glycosaminoglycan, amino acids, vitamin, mineral, and the like were isolated and identified from placenta, and unknown components are expected to be also included in the placenta.

On this background, the inventors of the present invention studied to develop a natural extract from which a steroid hormone is obtained in great quantities and which does not cause inherent side effects of steroid, and defined various medical uses of a placenta extract based on the fact that the placenta extract promotes muscular stem cell proliferation, hematopoietic stem cell proliferation, increases the number of blood cells and an amount of hemoglobin, and expedites differentiation from preadipocytes into adipocyte cells, thereby completing the present invention.

### DETAILED DESCRIPTION OF THE INVENTION

### Technical Problem

The present invention is to most efficiently extract steroid hormones comprising, for example, nandrolone, from discarded placenta of a mammal, such as swine, in great quantities for use as a medicament or health food for the prevention and treatment of human disease, or stock feed additives.

That is, the present invention provides a composition for the treatment and prevention of: a steroid hormonal disorder-induced disease selected from the group consisting of sexual dysfunction, osteoporosis, wasting muscular disorder, and aging; anemia disorder; or wound disorder, comprising a placenta extract as an active ingredient.

The present invention also provides a stock feed additive for controlling adipocytes differentiation, comprising a placenta extract as an active ingredient.

### Technical Solution

According to an aspect of the present invention, a composition includes a placenta extract as an active ingredient, wherein the placenta extract is obtained by extracting placenta with an extraction solvent selected from water, C1-4 alcohol, ethylacetate, chloroform, ether, hexane, dichloromethane, and a mixed solvent thereof.

In particular, the placenta extract according to the present invention may be used to treat or prevent disorder selected from: steroid hormonal disorder-induced disease selected from the group consisting of sexual dysfunction, osteoporosis, wasting muscular disorder, and aging; anemia disorder; and wound disorder selected from the group consisting of abrasion, laceration, bruise, incised wound, avulsion wound, penetrating wound, contusion, dislocation, sprain, gunshot wound, burn, frostbite, skin ulcer, xeroderma, keratoderma, crack, split, dermatitis, osteonecrosis, pain from dermatophytosis, surgical or vascular disorder wound, cornea wound, bedsore, decubitus, suture site after plastic surgery, wound from spinal injury, gynaecological wound, and chemical wound.

In addition, the placenta extract according to the present invention may promote differentiation from preadipocyte into adipocyte cells.

### Advantageous Effects

The placenta extract according to the present invention is a natural substance including high concentrations of anabolic steroid and sex hormone. The placenta extract may reduce side effects that may occur when typical chemically synthesized anabolic steroid and sex hormone drugs are used. In particular, the placenta extract may be used as men's contraceptives, anti-osteoporosis drugs, anti-anemic drugs, therapeutic agents for wasting diseases of muscular atrophy, agents for treating a decrease in men's generative functions, therapeutic agents for wounds, or adipocyte differentiation stimulating agents for the improvement of meat quality of livestock.

### DESCRIPTION OF THE DRAWINGS

FIG. 1 is a flowchart illustrating a method of preparing a placenta extract according to an embodiment of the present invention.

FIGS. 2 to 3 and 5 to 7 show, respectively, reference curves of estrone, estradiol, nandrolone, testosterone, and androstenedione assayed from a placenta extract according to an embodiment of the present invention.

FIG. 4 illustrates a plate used to analyze a nandrolone amount of a placenta extract according to an embodiment of the present invention.

FIG. 8 shows cell proliferation effects of a placenta extract according to an embodiment of the present invention.

FIG. 9 shows assay results of menstrual cycle effects of a placenta extract according to an embodiment of the present invention.

FIG. 10 shows assay results of the numerical change of intramedullary hematopoietic stem cells due to the treatment with a placenta extract according to an embodiment of the present invention.

FIG. 11 shows assay results of the numerical change of red blood cells in peripheral blood due to the treatment with a placenta extract according to an embodiment of the present invention.

FIG. 12 shows assay results of the quantitative change of hemoglobin in red blood cells due to the treatment with a placenta extract according to an embodiment of the present invention.

FIG. 13 shows biopsy results obtained by haematoxylin & eosin staining after the treatment with cream containing a placenta extract according to an embodiment of the present invention.

FIG. 14 shows adipocyte differentiation results of 3T3-L1 cells of a placenta extract according to an embodiment of the present invention.

FIG. 15 shows adipocyte differentiation results of mouse preadipocytes of a placenta extract according to an embodiment of the present invention.

FIG. 16 shows adipocyte differentiation results of mouse muscular stem cells of a placenta extract according to an embodiment of the present invention.

FIG. 17 shows adipocyte differentiation results of C2C12 cells of a placenta extract according to an embodiment of the present invention.

FIG. 18 shows adipocyte differentiation results of bovine muscular stem of a placenta extract according to an embodiment of the present invention.

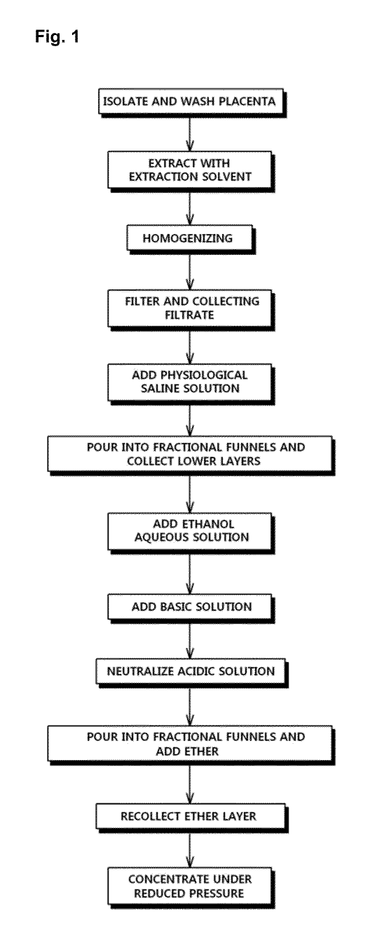

### BEST MODE

The present invention provides a composition includes a placenta extract as an active ingredient, wherein the placenta extract is obtained by extracting placenta with an extraction solvent selected from water, C1-4 alcohol, ethylacetate, chloroform, ether, hexane, dichloromethane and a mixed solvent thereof.

A placenta used according to the present invention includes a placenta of a mammal, such as swine, bovine, horse, or the like. For example, the placenta may be the placenta of swine.

The placenta extract may be obtained from a placenta by using a mixed solvent including C1-4 alcohol and chloroform.

The placenta extract may be obtained by the extraction with 10 to 20 mL of the extraction solvent per 1 g of placenta tissues. When the amount of the extraction solvent is outside the lower limit of the range, extraction efficiency may be low so that the content of a nandrolone, which is type of anabolic steroid, in the placenta extract may be low, and when the amount of the extraction solvent is outside the upper limit of the range, manufacturing costs may be too high.

The placenta extract may be isolated by, after the extraction with the extraction solvent, additional adding of a physiological saline solution. According to another embodiment of the present invention, the placenta extract may be isolated by, after the extraction with the extraction solvent, additional adding of a basic material, such as sodium hydroxide, and then, neutralizing with an acidic material, such as a sulfuric acid or a hydrochloric acid. According to another embodiment of the present invention, the placenta extract may be isolated by, after the extraction with the extraction solvent, additional adding of a physiological saline solution, adding of a basic material, such as sodium hydroxide, and then, neutralizing of the resultant solution with an acidic material, such as a sulfuric acid or a hydrochloric acid.

In detail, the placenta extract may be prepared by: adding an extraction solvent to placenta and homogenizing the result; filtering the homogenized product to remove the residual; adding a physiological saline solution to the filtrate and fractionizing the result solution into a lower layer and a supernatant; evaporating an organic solvent in the isolated lower layer; adding an aqueous alcohol solution to the residual solution; adding a basic material to the resultant solution, followed by heating in boiling water; adjusting a pH with an acidic material; adding an ether thereto and mixing the result, and performing layer-separation; and collecting, washing, and purifying the obtained upper ether layer.

The placenta extract may include, as a steroid hormone, nandrolone, testosterone, androstenedione, estradiol, estrone, and progesterone.

A composition according to an embodiment of the present invention may be provided in the form of any one of a pharmaceutical composition, health food, a medium additive, and a stock feed additive.

The composition according to an embodiment of the present invention may treat or prevent a steroid hormonal disorder-induced disease selected from the group consisting of sexual dysfunction, osteoporosis, wasting muscular disorder, and aging, and may be provided in the form of a pharmaceutical composition or health food.

In addition, the composition may treat or prevent anemia disorder, and may be provided in the form of a pharmaceutical composition or health food. In particular, the composition may effectively treat or prevent anemia disorder by at least one action of promoting hematopoietic stem cells proliferation, increasing the number of red blood cells, or increasing an amount of hemoglobin. In addition, the placenta extract does not inhibit a reproductive history of reproductive female, and may allow an embryo and fetus to wholly develop in pregnant female without negative effects.

In addition, the composition may treat or prevent wound disorder, and may be provided in the form of a pharmaceutical composition or health food. In particular, the composition may rapidly reduce a wound disorder site in an animal model having induced would disease. Examples of the wound disorder are abrasion, laceration, bruise, incised wound, avulsion wound, penetrating wound, contusion, dislocation, sprain, gunshot wound, burn, frostbite, skin ulcer, xeroderma, keratoderma, crack, split, dermatitis, osteonecrosis, pain from dermatophytosis, surgical or vascular disorder wound, cornea wound, bedsore, decubitus, suture site after plastic surgery, wound from spinal injury, gynaecological wound, and chemical wound.

A concentration of the placenta extract included in the composition according to the present invention may be in a range of 0.0001 to 30.0 wt %, for example, 0.0005 to 15.0 wt % based on the total weight of the composition. When the concentration of the placenta extract is less than 0.0001 wt %, distinctive effects may not be obtained, and when the concentration of the placenta extract is greater than 30.0 wt %, the concentration increase does not result in distinctive effects.

When the placenta extract according to the present invention is used as a pharmaceutical composition, any one of various methods that are publicly known in the pharmaceutical industry may be used. For example, the placenta extract may be mixed with a pharmaceutically acceptable support, excipient, diluent, or the like to be formulated as powder, granule, tablet, capsule, or injection, and may be non-orally administered, for example, intravenously, subcutaneously, intraperitoneally, or locally administered, or orally administered.

When the placenta extract according to the present invention is used as a pharmaceutical composition, a dosage may be appropriately determined according to the age, gender, body weight, health conditions, symptoms of disease, administration time, and administration method of a patient. For example, the dosage may be in a range of 0.01 to 100 mg/kg per day based on an adult.

In addition, the dosage of the placenta extract may increase or decrease according to administration pathway, disease severance, gender, body weight, age, or the like. Accordingly, the dosage does not limit the scope of the present invention.

The composition according to the present invention may be administered to a mammal, such as rats, mice, livestock, human beings, or the like, through various pathways. The administration method may be obvious, and for example, may be oral administration, or rectal or vein, muscle, subcutaneous, intrauterine dural, or intracerebroventricular injection.

In particular, when the placenta extract is administered to a human body, side effects may not occur compared to other synthetic medical products because the placenta extract is a natural extract and accordingly, safety of the placenta extract is guaranteed.

In addition, when the placenta extract according to the present invention is used as health food, the placenta extract may be provided in the form of powder, granule, tablet, capsule, syrup, or beverage. The health food may be used together with, in addition to the active ingredient, other foods or food additives, and may be appropriately used according to a conventional method. An amount of the active ingredient to be mixed may be appropriately determined according to purpose, for example, prevention, health maintenance, or therapeutic treatment.

In addition, the composition may be used as an additive for controlling adipocytes differentiation, and for example, either as a medium additive for promoting differentiation into adipocytes by incubating preadipocytes or muscular cells or as a stock feed additive for controlling adipocytes differentiation to improve meat quality of livestock. In particular, the placenta extract enables differentiation of, in addition to 3T3-L1, which is a preadipocyte strain, preadipocytes isolated from a mouse into adipocytes. In addition, the placenta extract enables differentiation of C2C12 cells, which is a muscular stem cell strain, and mouse and bovine muscular stem cells into adipocytes.

In addition, the present invention provides a method of inducing differentiation into adipocytes by incubating preadipocytes or muscular cells in a medium containing a medium additive for controlling adipocytes differentiation.

In addition, the present invention provides a method of screening an adipocytes differentiation controller, wherein the method includes incubating preadipocytes or muscular cells in a medium containing the medium additive for controlling adipocytes differentiation; treating a candidate material in the incubation product; and evaluating a level of differentiation into adipocytes.

The adipocytes differentiation controller may be an obesity medicine, a diabetes medicine, an anti-ageing drug, or an agent for enhancing meat quality of livestock.

In addition, the present invention provides a method of preparing the placenta extract, wherein the method includes: adding an extraction solvent to placenta and homogenizing the result; filtering the homogenized product to remove the residual; adding a physiological saline solution to the filtrate and fractionizing the result solution into a lower layer and a supernatant; evaporating an organic solvent in the isolated lower layer; adding an aqueous alcohol solution to the residual solution; adding a basic material to the resultant solution, followed by heating in boiling water; adjusting a pH with an acidic material; adding an ether thereto and mixing the result, and performing layer-separation; and collecting, washing, and purifying the obtained upper ether layer.

### MODE OF THE INVENTION

Hereinafter, the present invention is described in detail with examples. However, the present invention is not limited to the examples.

### Example 1

### Preparation of Placenta Extract

To most efficiently isolate steroid hormone in great quantities from swine placenta, amniotic fluids and sediments were completely removed from the swine placenta to obtain pure placenta. The obtained placenta was cut to a size of 10-30 g in advance to grind with a tissue homogenizer (Ultra-Turrax T25, IKA Co. USA), and the cut samples were frozen in a freezer at a temperature of −20° C.

As a solvent for extracting hormone of placenta, a mixed solution of chloroform (HPLC grade, SK Chemical Co. Seoul, Korea)/methanol (HPLC grade, Merck Co. Darmstadt, Germany) (50/50, v/v) was used. 25-50 g of placenta fragment was added to a 1000 mL beaker (Hanil Chemical Company, Seoul, Korea), and then, homogenized for 3 minutes by adding the mixed solution of chloroform/methanol thereto in an amount 8 times greater than that of the placenta fragment. When the homogenizing was completely performed, the resultant solution was filtered through Whatman No. 2 filtering paper to completely remove the residual remaining on the filtering paper.

The same amount of a 0.9% physiological saline solution was added to the prepared filtrate through a separation funnel (Hanil Chemical Company, Seoul, Korea), and then, smoothly shaken, and then, stood still for 10 minutes, and a lower layer was collected. From the collected material, an organic solvent was removed by evaporation by using a rotatory compressor, and then, the same amount of an ethanol/distilled water mixed solution (85/15, v/v) as that of the residual solution was added thereto and the mixture was homogeneously mixed.

Then, the same amount of 5M sodium hydroxide solution was added thereto and the mixture was heated in boiling water at a temperature of 80° C. for 45 minutes, followed by cooling at room temperature. A pH of the resultant solution was adjusted to be in a range of 2 to 3 using 6N sulfuric acid solution. The adjusted solution was divided and fractions thereof were placed in separation funnels, and ether in an amount a half of that of its corresponding solution was added thereto, followed by shaking to obtain a homogeneous solution, and standing still for 10 minutes to enable layer-separation. When layers are distinctively separated, the lower layers were placed in new separation funnels and a separation process was performed thereon. By doing so, a greater amount of extract was obtained. Thereafter, the lower layers were discarded, and the upper ether layers were collected and washed with distilled water, and only the ether layers were placed in a rotation decompression concentrator (1200 type, Eyela Co. Tokyo, Japan) for complete drying and concentrating. The concentrate was collected.

### Example 2

### Steroid Hormone Assay

2-1. Estrone Content Assay

Estrone content assay was performed as below by using estrone ELISA (DRG. EIA-4174).

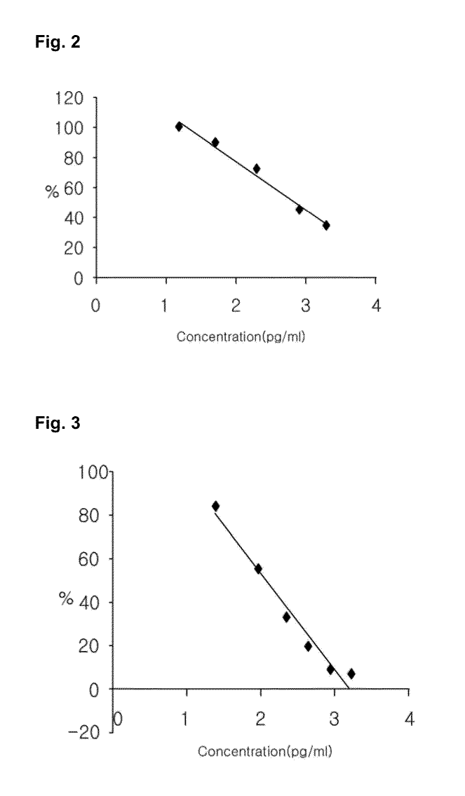

In detail, 50 μl of a control (stored at room temperature immediately before use, preservation at the temperature of 4° C.), 50 μl of a sample (diluted with tertiary distilled water), and 50 μl of a reference material (0, 15, 50, 200, 800, and 2000 pg/ml) were spread in each well, and 100 μl of an enzyme conjugate (stored at room temperature immediately before use, preservation at the temperature of 4° C.) was added thereto, and then, left for 1 hour at room temperature. Thereafter, the microtitre plate was washed four times with a washing buffer solution (40× concentration, diluted with distilled water, and 150 μl of substrate solution (stored at room temperature immediately before use, preservation at the temperature of 4° C.), and left for 30 minutes. After 30 minutes, 50 μl of a stop solution (stored at room temperature immediately before use, preservation at the temperature of 4° C.) was added thereto, and an optical density (O.D.) of the resultant product was measured at a wavelength of 450 nm. Results thereof are shown in Tables 1 and 2 and FIG. 2.

TABLE 1

| pg/ml | Reference material O.D |
|---|---|
| 0.00 | 1.97 |
| 15.00 | 1.72 |
| 50.00 | 1.52 |
| 200.00 | 1.17 |
| 800.00 | 0.77 |
| 2000.00 | 0.50 |

TABLE 2

| | | Extract | | |
|---|---|---|---|---|
| | | concentration | | |
| Sample | Dilution times | (ng/g) | Mean | ng/placenta g |
| Placenta 1 | 1000 | 47745 | 51218 | 512 |
| placenta 1 | 2000 | 54690 | 51218 | 512 |
| Placenta 2 | 2000 | 110124 | 122165 | 1099 |
| placenta 2 | 6000 | 134207 | 122165 | 1099 |
| Placenta 3 | 1000 | 50286 | 52322 | 837 |
| placenta 3 | 3000 | 54359 | 52322 | 837 |
| Placenta 4 | 1000 | 29556 | 34174 | 581 |
| placenta 4 | 2000 | 38791 | 34174 | 581 |
| Placenta 5 | 3000 | 246877 | 247239 | 2967 |
| | 6000 | 247602 | | |

2-2 Estradiol Content Assay

Estradiol content assay was performed as below by using estradiol ELISA (DRG. EIA-2693).

In detail, 25 μl of a control (stored at room temperature immediately before use, preservation at the temperature of 4° C.), 25 μl of a sample (diluted with tertiary distilled water), and 25 μl of a reference material (0, 25, 100, 250, 500, 1000, and 2000 pg/ml) were spread in each well, and 200 μl of an enzyme conjugate (stored at room temperature immediately before use, preservation at the temperature of 4° C.) was added thereto, and then, left for 1 hour at room temperature. Thereafter, the microtitre plate was washed three times with a washing buffer solution (40× concentration, diluted with distilled water, and 100 μl of substrate solution (stored at room temperature immediately before use, preservation at the temperature of 4° C.), and left for 15 minutes. After 15 minutes, 50 μl of a stop solution (stored at room temperature immediately before use, preservation at the temperature of 4° C.) was added thereto, and an optical density (O.D.) of the resultant product was measured at a wavelength of 450 nm. Results thereof are shown in Tables 3 and 4 and FIG. 3.

TABLE 3

| pg/ml | Reference material O.D |
|---|---|
| 0.00 | 2.02 |
| 25.00 | 1.75 |
| 100.00 | 1.02 |
| 250.00 | 0.47 |
| 500.00 | 0.29 |
| 1000.00 | 0.19 |
| 2000.00 | 0.11 |

TABLE 4

| | | Extract | | |
|---|---|---|---|---|
| | | concentration | | |
| Sample | Dilution times | (ng/g) | Mean | ng/placenta g |
| Placenta 1 | 2000 | 6085 | 6319 | 63 |
| Placenta 1 | 6000 | 6553 | 6319 | 63 |
| Placenta 2 | 2000 | 9515 | 9494 | 85 |
| Placenta 2 | 6000 | 9473 | 9494 | 85 |
| Placenta 3 | 2000 | 7659 | 8197 | 131 |
| Placenta 3 | 6000 | 8735 | 8197 | 131 |
| Placenta 4 | 2000 | 5396 | 5743 | 98 |
| Placenta 4 | 6000 | 6090 | 5743 | 98 |
| Placenta 5 | 2000 | 18385 | 17992 | 216 |
| | 6000 | 17600 | | |

2-3 Nandrolone(19-Nortestosterone) Content Assay

Nandrolone content assay was performed as below by using 19-nortestosterone-EIA (Euro-Diagnostica B. V. 5082NOR1p).

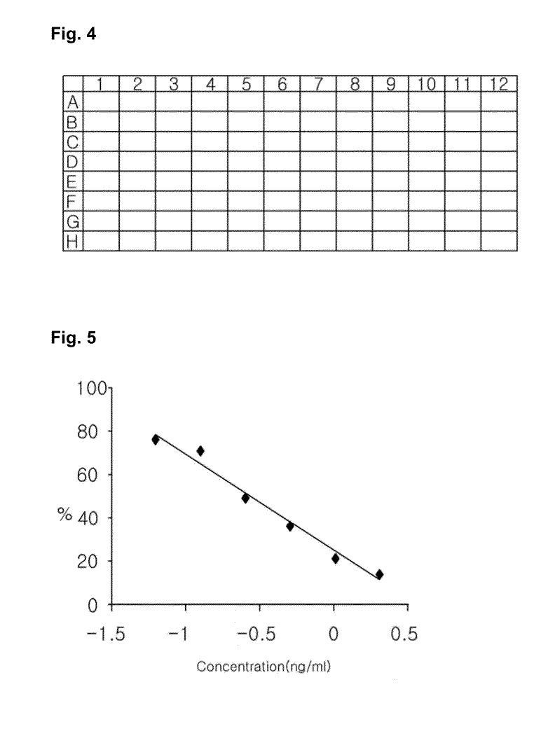

In detail, 100 μl of zero standard was added to A1 well of the microtitre plate of FIG. 4, and then, 50 μl of zero standard was added to B1 well, and then, standards 1-6 were respectively added to B, C, D, E, F, G H 1 wells. 50 μl of an assay sample (diluted with tertiary distilled water) was added to the remaining wells, and 25 μl of an enzyme conjugate solution (in dark, at room temperature before use, stored at the temperature of 4° C., for long-term use, at the temperature of −20° C.) and 25 μl of an antibody solution (stored at the temperature of 4° C., power state, dissolved with 4 ml of dilution buffer) were added to wells (standard and sample wells) other than A1 well. The resultant plate was placed in dark by shielding light with silver foil, and left at the temperature of 4° C. for 1 hour, and washed three times with a washing buffer solution. 100 μl of substrate solution (in dark, stored at room temperature before use, for long-term use, stored at the temperature of 4° C.) was added to the washed plate, and then, the plate was left for 30 minutes, and then, 100 μl of a stop solution was added thereto and optical density (O.D.) thereof was measured at a wavelength of 450 nm. Results thereof are shown in Tables 5 and 6 and FIG. 5.

TABLE 5

| ng/ml | Reference material O.D |
|---|---|
| zero | 0.075 |
| zero′ | 0.67 |
| 0.0625 | 0.63 |
| 0.125 | 0.54 |
| 0.25 | 0.45 |
| 0.5 | 0.35 |
| 1 | 0.26 |
| 2 | 0.17 |

TABLE 6

| | | Extract | | |
|---|---|---|---|---|
| | | concentration | | |
| Sample | Dilution times | (ng/g) | Mean | ng/placenta g |
| Placenta 1 | 1000 | 43097 | 41384 | 414 |
| Placenta 1 | 3000 | 39672 | 41384 | 414 |
| Placenta 2 | 1000 | 35218 | 34879 | 314 |
| Placenta 2 | 4000 | 34540 | 34879 | 314 |
| Placenta 3 | 1000 | 30783 | 29774 | 476 |
| Placenta 3 | 3000 | 28764 | 29774 | 476 |
| Placenta 4 | 1000 | 34695 | 35893 | 610 |
| Placenta 4 | 3000 | 37090 | 35893 | 610 |
| Placenta 5 | 1000 | 38813 | 39392 | 473 |
| | 3000 | 39970 | | |

2-4 Testosterone Content Assay

Testosterone content assay was performed as below by using testosterone ELISA (DRG. EIA-1559).

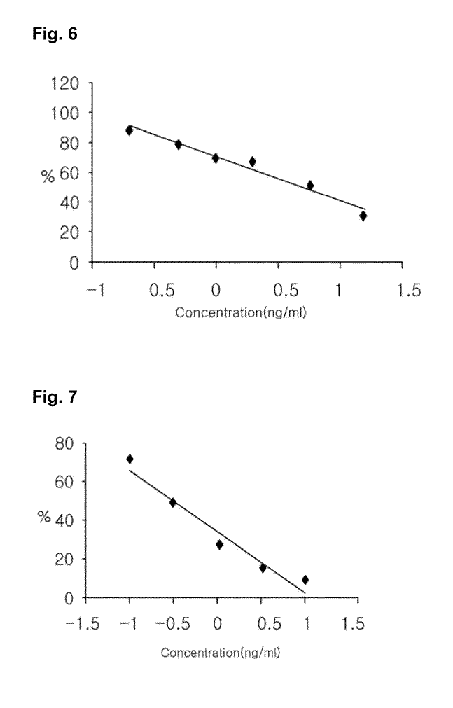

In detail, 50 μl of a control (stored at room temperature immediately before use, preservation at the temperature of 4° C.), 50 μl of a sample (diluted with tertiary distilled water), and 50 μl of a reference material (0, 0.2, 0.5, 1, 2, 6, and 16 ng/ml) were spread in each well, and 100 μl of an enzyme conjugate (stored at room temperature immediately before use, preservation at the temperature of 4° C.) was added thereto, and then, left for 1 hour at room temperature. Thereafter, the microtitre plate was washed three times with a washing buffer solution (40× concentration, diluted with distilled water, and 150 μl of substrate solution (stored at room temperature immediately before use, preservation at the temperature of 4° C.) was added to the washed plate, and the plate was left for 30 minutes. After 30 minutes, 100 μl of a stop solution (stored at room temperature immediately before use, preservation at the temperature of 4° C.) was added thereto, and an optical density (O.D.) of the resultant product was measured at a wavelength of 450 nm. Results thereof are shown in Tables 7 and 8 and FIG. 6.

TABLE 7

| ng/ml | Reference material O.D |
|---|---|
| 0 | 2.33 |
| 0.2 | 2.08 |
| 0.5 | 1.83 |
| 1 | 1.52 |
| 2 | 1.14 |
| 6 | 0.62 |
| 16 | 0.30 |

TABLE 8

| | | Extract | | |
|---|---|---|---|---|
| | | concentration | | |
| Sample | Dilution times | (ng/g) | Mean | ng/placenta g |
| placenta 1 | 10 | 648 | 655 | 7 |
| placenta 1 | 30 | 663 | 655 | 7 |
| placenta 2 | 10 | 1147 | 1202 | 11 |
| placenta 2 | 30 | 1256 | 1202 | 11 |
| placenta 3 | 10 | 461 | 470 | 8 |
| placenta 3 | 30 | 480 | 470 | 8 |
| placenta 4 | 10 | 2668 | 2872 | 49 |
| placenta 4 | 30 | 3076 | 2872 | 49 |
| placenta 5 | 10 | 1546 | 1548 | 18 |
| | 30 | 1550 | | |

2-5 Androstenedione Content Assay

Androstenedione content assay was performed as below by using androstenedione ELISA (DRG. EIA-1559).

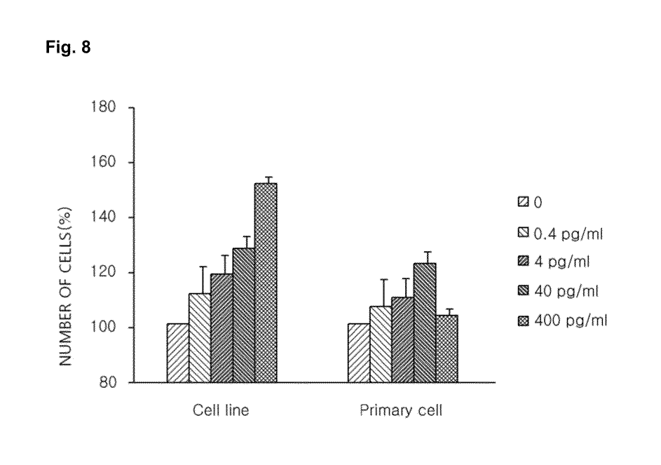

In detail, 20 μl of a control (stored at room temperature immediately before use, preservation at the temperature of 4° C.), 20 μl of a sample (diluted with tertiary distilled water), and 20 μl of a reference material (0, 0.2, 0.5, 1, 2, 6, and 16 ng/ml) were spread in each well, and 200 μl of an enzyme conjugate (stored at room temperature immediately before use, preservation at the temperature of 4° C.) was added thereto, and then, left for 1 hour at room temperature. Thereafter, the microtitre plate was washed three times with a washing buffer solution (40× concentration, diluted with distilled water, and 200 μl of substrate solution (stored at room temperature immediately before use, preservation at the temperature of 4° C.) was added to the plate, and the plate was left for 15 minutes. After 15 minutes, 50 μl of a stop solution (stored at room temperature immediately before use, preservation at the temperature of 4° C.) was added thereto, and an optical density (O.D.) of the resultant product was measured at a wavelength of 450 nm. Results thereof are shown in Tables 9 and 10 and FIG. 7.

TABLE 9

| ng/ml | Reference material O.D |
|---|---|
| 0.00 | 1.87 |
| 0.10 | 1.09 |
| 0.30 | 0.87 |
| 1.00 | 0.59 |
| 3.00 | 0.21 |
| 10.00 | 0.09 |

TABLE 10

| | | Extract | | |
|---|---|---|---|---|
| | | concentration | | |
| Sample | Dilution times | (ng/g) | Mean | ng/placenta g |
| Placenta 1 | 1000 | 3858 | 3625 | 36 |
| Placenta 1 | 3000 | 3392 | 3625 | 36 |
| Placenta 2 | 1000 | 4046 | 3726 | 34 |
| Placenta 2 | 3000 | 3406 | 3726 | 34 |
| Placenta 3 | 1000 | 2848 | 2881 | 46 |
| Placenta 3 | 3000 | 2914 | 2881 | 46 |
| Placenta 4 | 1000 | 2425 | 2391 | 41 |
| Placenta 4 | 3000 | 2356 | 2391 | 41 |
| Placenta 5 | 1000 | 2436 | 2443 | 29 |
| | 3000 | 2450 | | |

Steroid hormone extracted from 1 g of swine placenta by using various methods according to the examples was isolated and purified, and results thereof are shown in Table 11 below.

TABLE 11

| | Placenta | Placenta | Placenta | Placenta | Placenta |
|---|---|---|---|---|---|
| | 1 | 2 | 3 | 4 | 5 |
| Nandrolone (ng/g) | 414 | 314 | 476 | 610 | 473 |
| Testosterone (ng/g) | 7 | 11 | 8 | 49 | 19 |
| Androstenedione (ng/g) | 36 | 34 | 46 | 41 | 29 |
| Estradiol (ng/g) | 63 | 85 | 131 | 98 | 216 |
| Estrone (ng/g) | 512 | 1099 | 837 | 581 | 2967 |

### Experimental Example 1

### Proliferation Effects of Muscular Cells

To confirm effects of the placenta extracts prepared according to the examples on muscular stem cells (cell strain, initial cultured cells), the placenta 1 extract prepared according to the example was added in amounts of 0, 0.4, 4, 40, and 400 pg/ml based on nandrolone to DMEM, and incubated in a 5% CO₂, 37° C. incubator. Three days after the incubation, MTT assay was performed on each well by using an ELISA reader to measure adsorption at a wavelength of 540 nm.

As a result, as illustrated in FIG. 8, when the incubating was performed with the extract, proliferation of muscular cells was promoted concentration-dependently compared to the control that was not treated with the extract. Accordingly, it was confirmed that the placenta extract according to the present invention is safe for cell incubation and is effective for proliferation of muscular cells.

### Experimental Example 2

### Anemia Treatment Effects Identification

The placenta 1 extract prepared according to the example was dissolved with 100% ethanol as a primary solvent, and sesame oil for body injection was added thereto to obtain a 1 g/ml concentration, and the resultant product was diluted into 0.1% concentration. Herein, 100% was defined as a case in which the extract was diluted with sesame oil by a factor of 100. The diluted extract was subcutaneously injected for 14 days to 6-8 week-old CD-1 mice which had been raised under physiological conditions (6 mice per each group).

By doing so, effects of the placenta extract (Placenta Steroid Extracts; PSC) on an estrous cycle, change of hematopoietic stem cells, change of the number of red blood cells, and a quantitative change of hemoglobin in red blood cells were confirmed as below.

2-1. Reproductive Cycle Change Assay

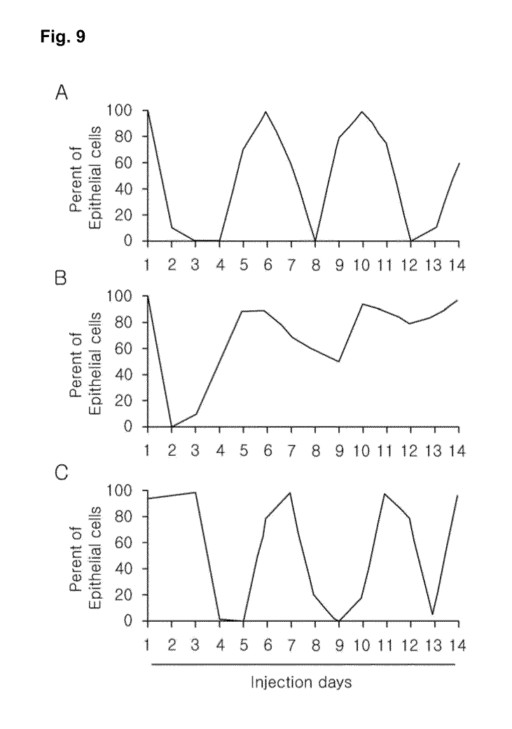

To identify effects of the placenta extract on a reproductive cycle, a reproductive cycle was assayed by vaginal smearing during the administration of the placenta extract. As illustrated in 9B, when only nandrolone was administered, a reproductive cycle gradually became non-periodic. However, when the placenta extract was used (see FIG. 9C), similar to the control treated with sesame oil (see FIG. 9A), a periodical reproductive cycle was obtained. Based on these results, it was confirmed that the placenta extract does not damage periodic characteristics of the reproductive cycle.

2-2. CD34-Marked Numerical Change Assay of Hematopoietic Stem Cells Using FACS

Whether the placenta extract promotes proliferation of hematopoietic stem cells was identified with CD34 that is a marking factor. CD-1 mouse treated with 0.1% placenta extract was sacrificed to collect a thighbone, and then, a bone marrow was extracted therefrom by using PBS solution. Thereafter, the bone marrow was reacted with CD34 specific antibody, and then, CD34 hematopoietic stem cells were counted by using a fluorescent activated cell sorter.

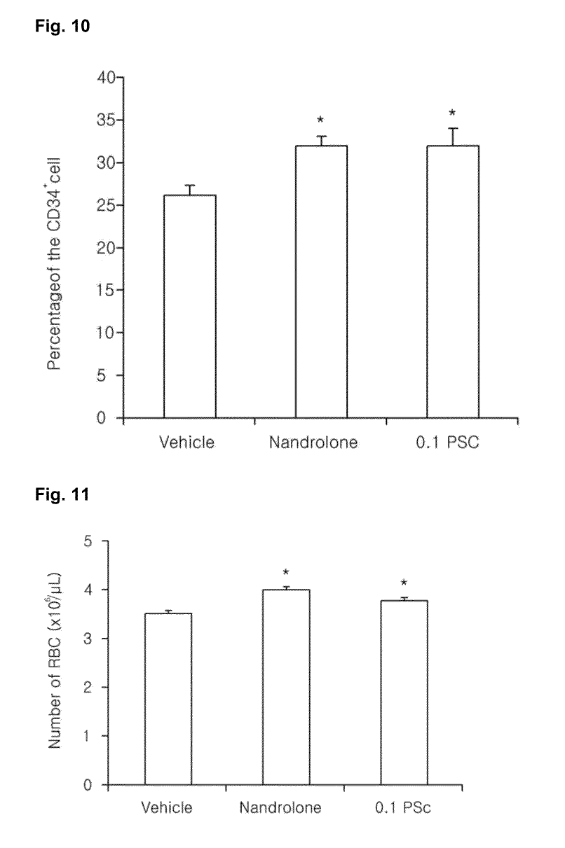

As a result, as illustrated in FIG. 10, regarding the group treated with nandrolone, which was conventionally used as anemia medicine, the number of hematopoietic stem cells significantly increased, and in addition, even in the group treated with 0.1% placenta extract, the number of the cells significantly increased. In comparison of nandrolone with the placenta extract, although the placenta extract produced smaller effects than nandrolone, the placenta extract significantly induced proliferation of hematopoietic stem cells.

2-3. Numerical Change Assay of Red Blood Cells in Peripheral Blood

The cells were treated with 0.1% placenta extract under the same conditions as described above, and then, peripheral blood was collected, and then, 1 mg/ml of EDTA was added thereto to prevent blood coagulation. Then, the number of red blood cells was counted by using an analyzer.

As a result, as illustrated in FIG. 11, it was confirmed that the number of red blood cells significantly increased in the groups treated with nandrolone and placenta extract compared to the control. Such results show that the placenta extract directly acts on the treatment of anemia.

2-4. Quantitative Analysis of Hemoglobin in Red Blood Cells

Quantitative analysis of hemoglobin in red blood cells in blood obtained as described above was performed by using poah100i (Sysmex).

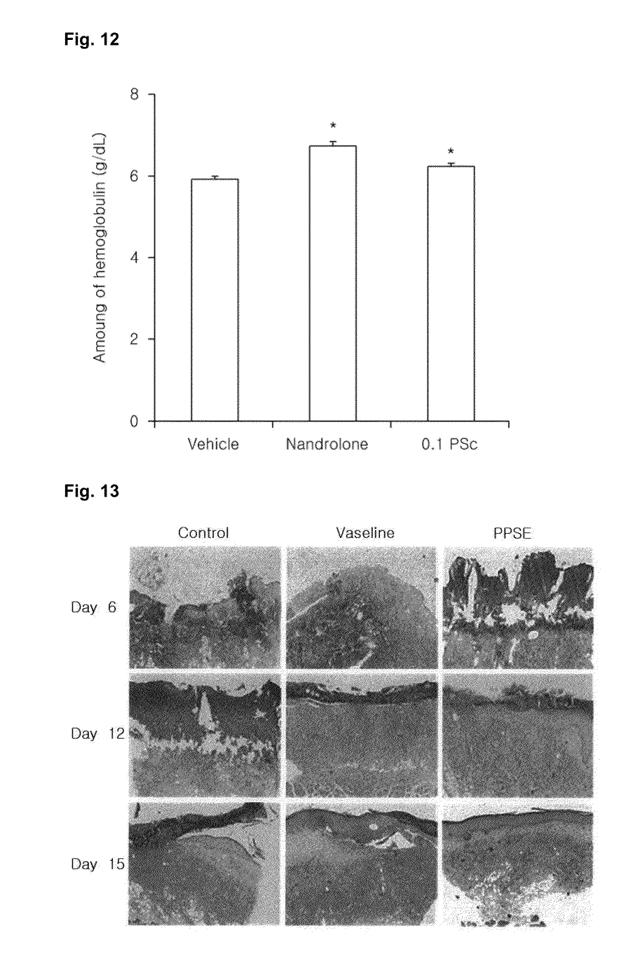

As a result, as illustrated in FIG. 12, it was confirmed that the quantity of hemoglobin in red blood cells significantly increased in the group treated with 0.1% placenta extract. Such a result shows that the placenta extract is a very useful material in treating anemia

### Experimental Example 3

### Wound Treatment Effects Identification

3-1. Preparation of Placenta Extract Cream

15 g of polysorbate was added to 200 g of distilled water while heating, and dispersed by stirring, and then, 8 g of the placenta 1 extract prepared according to the example was dissolved and then added thereto, thereby preparing a water-phase preparation liquid. In addition, while heating, 5 g of disodium hydrogen phosphate and 0.5 g of sodium acetate were sequentially added thereto and dissolved to prepare an oil-phase preparation liquid.

Prior to heating, the water-phase preparation liquid was added to the oil-phase preparation liquid and the liquids were mixed to be miscible, and then, an appropriate amount of distilled water was added thereto until the total amount reached 1 kg wt. Then, homogenizing was performed thereon at a rate of 10,000 rpm or more while cooling, thereby preparing a cream. In this regard, as an additive, acetyl alcohol, stearyl alcohol, isopropyl myristate, propylene glycol, or wax was used.

3-2. Wound Disorder Treatment Effects Identification

To identify effects of the placenta extract cream prepared according to Experimental Example 3-1 on wound disorder, wound disorder was induced in 10-week old male rat, and then, the placenta extract cream prepared according to Experimental Example 3-1 was applied thereon in an amount of 1 g two times per day. On 6th, 12th, and 15th days after the cream treatment, the rat was sacrificed to collect tissues of wound disorder site. While being careful not to deform the collected tissue, the tissue was fixed with 10% formaldehyde.

To perform histological examination, tissues were fixed with 10% neutral formalin for one to two days and then, embedded with paraffin, sliced to a thickness of 4 μm, and then attached to a probe-on plus slide (Fisher Scientific, USA) to which an organosaline was attached, followed by treatment in a warmer at a temperature of 56° C. for 30 minutes. To deparaffinize the tissues, they were fixed three times with xylene for 5 minutes. Then, a water immersion process was performed thereon with 100%, 90% and 75% ethanol each for 3 minutes, followed by washing with a tris buffer solution for 10 minutes. The resultant tissues were stained with haematoxylin & eosin (H&E), and encapsulation was performed thereon with crystal mount to prevent any loss of tissue specimen, and the result was identified with a microscope.

As a result, as illustrated in FIG. 13, in the case of the control, on the sixth day, epidermis containing severe wound was not yet recovered, on the 12th day, the epidermis became very thick and thus, it was considered that a scar was formed, on the 15th day, the scar formation was slightly reduced to but it cannot be said that the wound was recovered into normal tissues, and in particular, dermis contained many immune cells.

In addition, in the case of the group treated with vaseline as a positive control group, on the 12th day, the scar formation was much reduced compared to the control, and on the 15th day, the tissues were much recovered into normal tissues compared to the control. However, like the control, dermis contained many immune cells.

Meanwhile, in the case of the placenta extract treated group (PPSE), on the 6th day, the scar formation level was similar to that of the 12th-day control, and on the 12th day, the wound was much recovered, and on the 15th day, tissues were almost recovered into normal tissues. In particular, dermis contained a minimum number of immune cells.

### Experimental Example 4

### Adipocyte Differentiation Induction Identification

4-1. Adipocyte Differentiation Induction of Preadipocvtes

Preadipocytes were in-vitro incubated with 10% FBS, 1% penicilin-streptomycin, and 4 mM L-glutamin supplemented DMEM/high glucose (HyClone) culture as basic culture, under conditions including 5% CO₂ and 37° C., on a 6-well culture plate. When the cells 80% confluently reached, adipocyte differentiation was performed thereon. Adipocyte differentiation was induced as follows: 1) adipocyte differentiator (DS: 10 μg/ml insulin, 1 μM dexamethasone, and 0.5 mM 3-isobutyl-1-methylxantine) was added to DMEM and 10% FBS, and two days after the differentiation, only 10 μg/ml insulin was added thereto, and the cells were incubated, and 2) the placenta 1 extract (PTSE) prepared according to the examples was added to induce adipocyte differentiation while the culture solution was refreshed at intervals of 3 days. 4 days after the incubation, adipocytes differentiation was identified by oil-red-O staining under microscope, and after oil-red-O stained by adding 100% isopropanol was extracted for quantification, adsorption was measured using ELISA (Molecular Devices, USA) at a wavelength of 510 nm.

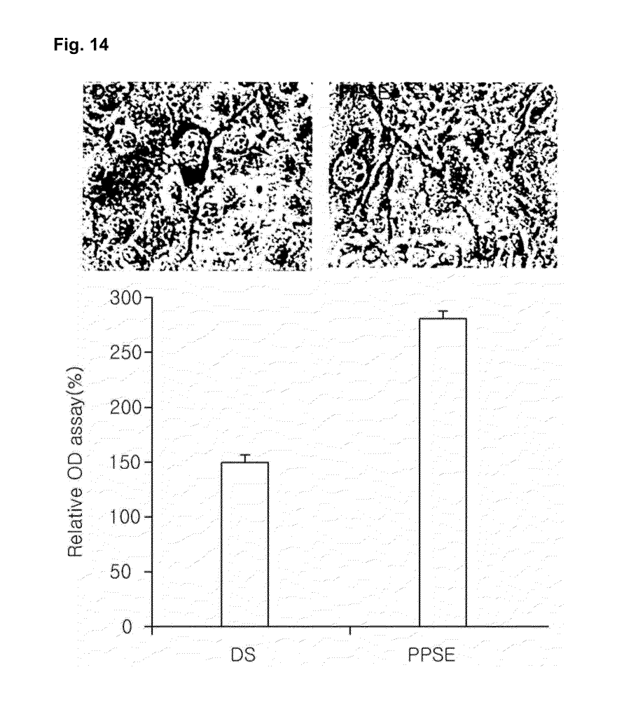

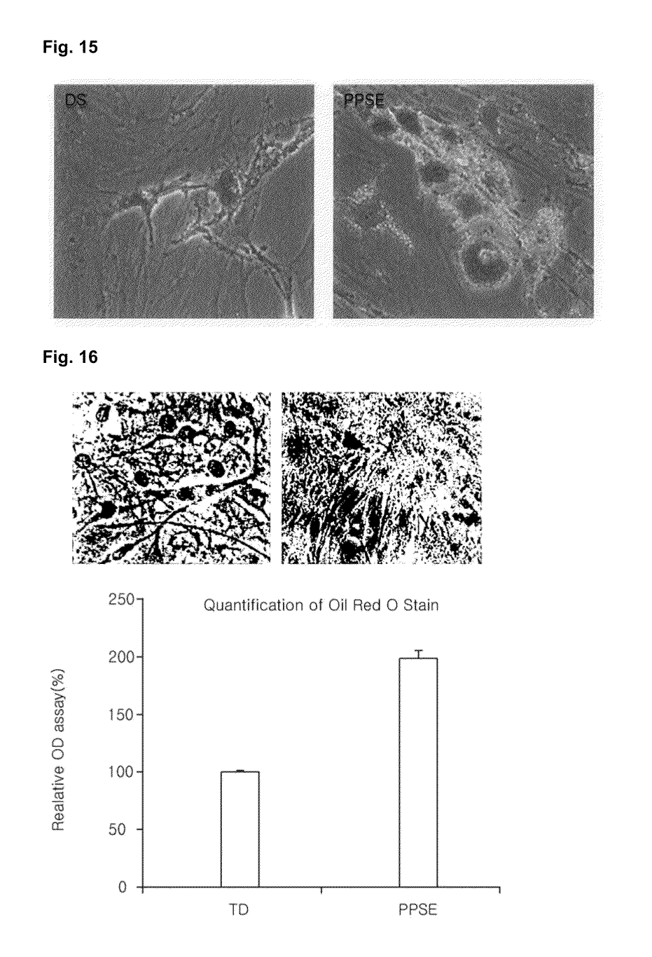

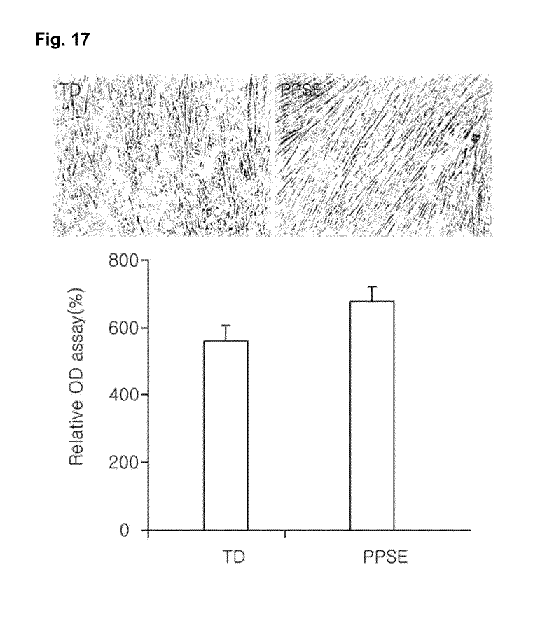

As a result, as shown in FIGS. 14 and 15, it was confirmed that the placenta extract produced more red stained fat drops than adipocyte differentiator.

4-2. Adipocyte Differentiation Induction of Muscular Cells

Muscular cells were in-vitro incubated with 10% FBS, 1% penicilin-streptomycin, and 4 mM L-glutamin supplemented DMEM/high glucose (HyClone) culture as basic culture, under conditions including 5% CO₂ and 37° C., on a 6-well culture plate. When the cells 80% confluently reached, adipocyte differentiation was performed thereon. Adipocyte differentiation was induced as follows: 1) a hetero differentiator [TD: 10 μg insulin, 10 μM dexamethasone, 0.5 mM 3-isobutyl-1-methylxantine (IBMX), 200 μM ascorbic acid, 33 μM biotin, and 1 mM capric acid (Sigma chemical Co.)] and 2) swine placenta extract (PPSE) were added to a basic culture solution and, the culture solution was refreshed at intervals of 3 days to induce adipocyte differentiation. 6 days after the incubation, adipocytes differentiation was identified by oil-red-O staining under microscope, and after oil-red-O stained by adding 100% isopropanol was extracted for quantification, adsorption was measured using ELISA (Molecular Devices, USA) at a wavelength of 510 nm.

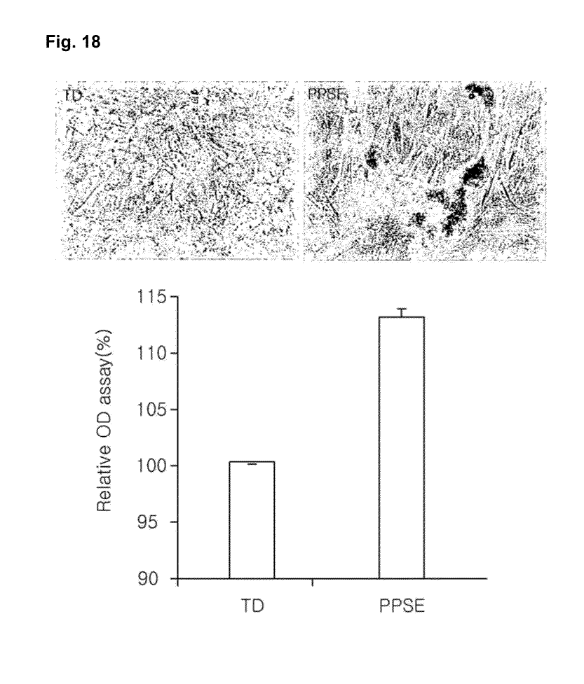

As a result, as shown in FIGS. 16 and 18, it was confirmed that the placenta extract produced more red stained fat drops than adipocyte differentiator.

1. A composition comprising a placenta extract as an active ingredient, wherein the placenta extract is obtained by extracting placenta with an extraction solvent selected from the group consisting of water, C1-4 alcohol, ethylacetate, chloroform, ether, hexane, dichloromethane, and a mixed solvent thereof.

2. The composition of claim 1, wherein the placenta extract is prepared by extracting placenta with a mixed solvent comprising C1-4 alcohol and chloroform.

3. The composition of claim 1, wherein, after the extracting of placenta with the extraction solvent, the placenta extract is isolated by additional adding of a physiological saline solution.

4. The composition of claim 1, wherein, after the extracting of placenta with the extraction solvent, the placenta extract is isolated by additional adding of a basic material and then, neutralizing with an acidic material.

5. The composition of claim 1, wherein, after the extracting of placenta with the extraction solvent, the placenta extract is isolated by adding of a physiological saline solution, adding of a basic material, and then, neutralizing with an acidic material.

6. The composition of claim 1, wherein the placenta extract is prepared by: adding an extraction solvent to placenta and homogenizing the result; filtering the homogenized product to remove the residual; adding a physiological saline solution to the filtrate and fractionizing the result solution into a lower layer and a supernatant; evaporating an organic solvent in the isolated lower layer; adding an aqueous alcohol solution to the residual solution; adding a basic material to the resultant solution, followed by heating in boiling water; adjusting a pH with an acidic material; adding an ether thereto and mixing the result, and performing layer-separation; and collecting, washing, and purifying the obtained upper ether layer.

7. The composition of claim 1, wherein the placenta extract comprises, as a steroid hormone, nandrolone, testosterone, androstenedione, estradiol, estrone, and progesterone.

8. The composition of claim 1, wherein the composition is any one of a pharmaceutical composition, health food, a medium additive, or a stock feed additive.

9. The composition of claim 8, wherein the composition treats or prevents a steroid hormonal disorder-induced disease selected from the group consisting of sexual dysfunction, osteoporosis, wasting muscular disorder, and aging.

10. The composition of claim 8, wherein the composition treats or prevents anemia disorder.

11. The composition of claim 10, wherein the composition treats or prevents anemia disorder by at least one action of promoting hematopoietic stem cells proliferation, increasing the number of red blood cells, and increasing an amount of hemoglobin.

12. The composition of claim 8, wherein the composition treats or prevents wound disorder.

13. The composition of claim 12, wherein the wound disorder comprises abrasion, laceration, bruise, incised wound, avulsion wound, penetrating wound, contusion, dislocation, sprain, gunshot wound, burn, frostbite, skin ulcer, xeroderma, keratoderma, crack, split, dermatitis, osteonecrosis, pain from dermatophytosis, surgical or vascular disorder wound, cornea wound, bedsore, decubitus, suture site after plastic surgery, wound from spinal injury, gynaecological wound, or chemical wound.

14. The composition of claim 8, wherein the composition is a medium additive or a stock feed additive for controlling adipocytes differentiation.

15. The composition of claim 14, wherein the composition is a medium additive for promoting differentiation into adipocytes by incubating preadipocytes or muscular cells.

16. The composition of claim 14, wherein the composition is a stock feed additive for controlling adipocytes differentiation to improve meat quality of livestock.

17. A method of preparing a placenta extract, comprising:
adding an extraction solvent to placenta and homogenizing the result;
filtering the homogenized product to remove the residual;
adding a physiological saline solution to the filtrate and fractionizing the result solution into a lower layer and a supernatant;
evaporating an organic solvent in the isolated lower layer;
adding an aqueous alcohol solution to the residual solution;
adding a basic material to the resultant solution, followed by heating in boiling water;
adjusting a pH with an acidic material;
adding an ether thereto and mixing the result, and performing layer-separation; and
collecting, washing, and purifying the obtained upper ether layer.
